(19) United States
(12) Patent Application Publication
(10) Pub. No.: US 2024/0046542 A1
(43) Pub. Date: Feb. 8, 2024

(54) Automatic human body parameter generation method based on machine learning

(71) Applicant: SHANGHAI LINCTEX DIGITAL TECHNOLOGY CO, LTD., Shanghai (CN)

(72) Inventors: Likang Luo, Shanghai (CN); Luyuan Wang, Shanghai (CN); Xiaogang Jing, Shanghai (CN); Chen Liu, Shanghai (CN); Ninghai Huang, Shanghai (CN); Zexi Shao, Shanghai (CN)

(21) Appl. No.: 18/490,722

(22) Filed: Oct. 19, 2023

Related U.S. Application Data
(63) Continuation-in-part of application No. 17/520,595, filed on Nov. 5, 2021.
Application No. 17/520,595 is a continuation of application No. PCT/CN2019/105296, filed on Sep. 11, 2019.

(30) Foreign Application Priority Data
May 7, 2019 (CN) 201910414893.7

(51) Int. Cl. G06T 13/40; G16H 10/20; G06N 20/20

(52) U.S. Cl. CPC G06T 13/40; G16H 10/20; G06N 20/20

(57) ABSTRACT

A method of automatically generating human-body parameters using machine learning, including the following steps: initializing a converting program, inputs of which are accurate human-body parameters and outputs of which are general body-shape descriptions; inputting several groups of accurate human-body parameters into the converting program, so as to obtain various combinations of general body-shape descriptions, which are to be used as training sets for subsequent steps; carrying out training through machine learning by using the previously obtained training sets, to obtain a mapping relationship between the general body-shape descriptions and parameters of a 3D human body model; recording gender, height, and weight information from a user and the user's responses to a series of preset general descriptive questions about body shape, and using the previously obtained mapping relationship, to output accurate human-body parameters representing an actual human body of the user.

### CROSS-REFERENCE TO RELATED APPLICATIONS

This application is a Continuation-in-part of application Ser. No. 17/981,137. This application claims priorities from application Ser. No. 17/981,137 filed Nov. 4, 2022, PCT Application No. PCT/CN2019/105296 filed Sep. 11, 2019, and from the Chinese patent application 201910414893.7 filed May 7, 2019, the contents of which are incorporated herein in the entirety by reference.

### TECHNICAL FIELD

The invention relates to the field of 3D human body reconstruction technology, in particular to an automatic human body parameter generation method based on machine learning.

### BACKGROUND TECHNOLOGY

During virtual dressing, it is often the case that a 3D human body model in line with the user's body shape shall be generated for the model to wear the garment and test the dressing effect. In most of the current 3D human body reconstruction methods, devices like the depth camera are utilized to scan the bodies of real users, and the 3D human body model is reconstructed based on the information obtained. This method is found with three defects: first, special devices are required to collect the human body information, which increases the device costs; second, the sensors shall be placed in an open and unblocked environment, restricting the site to some extent; third, the users shall pose as instructed to allow for rotational or multi-angle photographing so as to collect human body data, requiring some skills and even becoming an obstacle for some users.

In recent years, the development of machine learning greatly promotes the advancement of all computer science fields, leading to lots of open-sourced 3D model datasets about human bodies. The parameter mapping relationship of people with different body shapes can be obtained by means of machine learning, and it just takes some learning and training costs to efficiently get accurate results in future real applications, providing a new thought to the reconstruction of the 3D human body model.

### SUMMARY OF THE INVENTION

The invention aims to provide an automatic human body parameter generation method based on machine learning. The simple, efficient, and low-cost method provided by this invention can be utilized to rapidly generate accurate human body parameters close to the user's real body shape after inputting basic information about the user and answering the predefined questions.

An automatic human body parameter generation method based on machine learning, comprising the following steps:
(1) Initialize a converting program, with accurate human body parameters as the inputs and general body shape descriptions as the outputs. input several groups of accurate human body parameters into the said converting program in Step (1) to get different combinations of general body shape descriptions as the training sets for subsequent steps;
(2) Train by using the training sets from Step (1) through machine learning to get mapping relationship between general body shape descriptions and 3D human body model parameters;
(3) By inputting gender, specific height, and weight information, the users answer a series of preset general body shape-related descriptive questions (“yes” or “no”), and utilize the mapping relationship from Step (2) to rapidly output accurate human body parameters in line with the actual situation of the users.

In the said Step (1), the accurate data of the human body's different parts are within a certain range; with male neck shape as an example, the general body shape description is set in the converting program: when the neck circumference inputted is not more than 35 cm, the neck shape is “slightly thin”; when falling within 35-40 cm, the neck shape is “normal”; when greater than 40 cm, the neck shape is “slightly thick”. Likewise, with male waist shape as an example, the following general body shape description is presented in the converting program: when the waist-to-hip ratio is not more than 0.8, the waist shape is “sunken”; when greater than 0.8 and not more than 0.87, the waist shape is “straight”; when greater than 0.87 and not more than 0.93, the waist shape is “generally protruding”. In this way, all human body parameters inputted can be converted to get a group of general body shape descriptions about the human body model, namely, a group of answers to the body shape-related descriptive questions.

Further, for a certain group of human body measurements, a group of general human body descriptions can be outputted with the help of the converting program, such as “normal” neck shape, chest shape with “severely muscular”, “regular” shoulder shape, “straight” back, “slightly short” arm length, “generally protruding” waist shape, “flat” abdomen shape, “inverted triangular” body shape, “medium-sized” skeleton, and “normal” leg shape.

Further, when the model is being used in real life, the user shall answer a group of predefined body shape-related descriptive questions to get general body shape descriptions about the user.

In the said Step (3), every 3D human body model is equipped with a group of human body measurements; to get a 3D human body model in line with the user's real body shape, general body shape descriptions given by the user shall be correlated with human body measurements of corresponding body shapes, which are called mapping relationship.

### DESCRIPTION OF FIGURES

FIG. 1 presents some general descriptions and judgment conditions in the converting program;

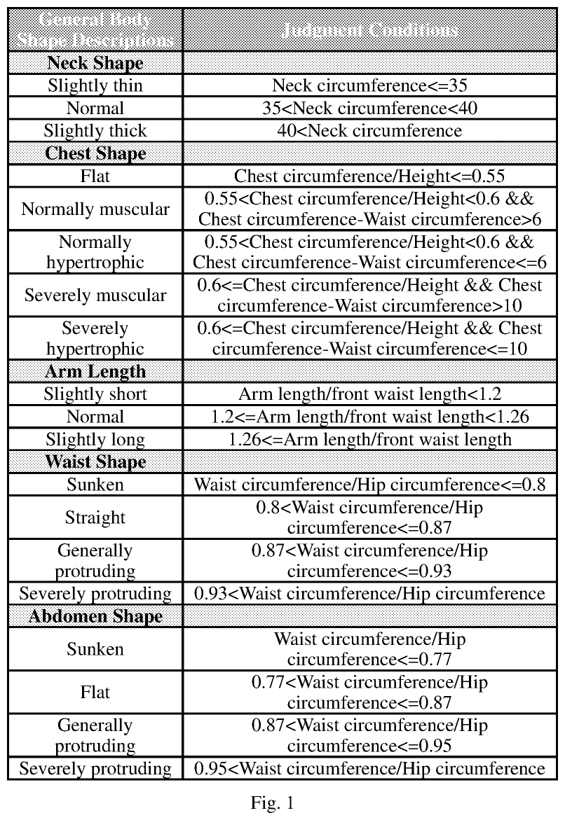

FIG. 2 presents some predefined questions (about females) on general human body descriptions provided in this invention;

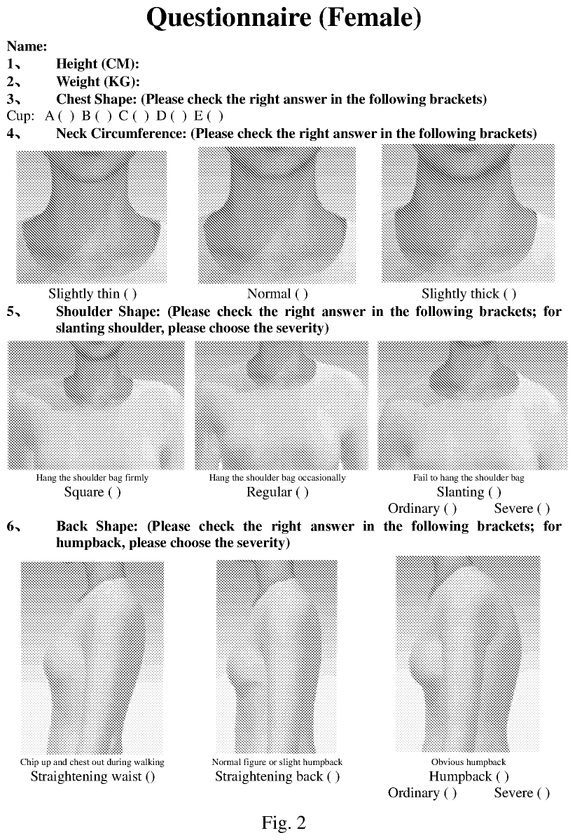

### DETAILED DESCRIPTION OF THE INVENTION EMBODIMENTS

Next, the technical solution in this invention will be further detailed in conjunction with figures and embodiments.
(1) Initialize a converting program, with accurate human body parameters as the inputs and general body shape descriptions as the outputs; input several groups of accurate human body parameters into the converting program to get different combinations of general body shape descriptions as the training sets for subsequent steps;
(2) Train by using the training sets from Step (1) through machine learning to get mapping relationship between general body shape descriptions and 3D human body model parameters;
(3) By inputting gender, specific height, and weight information, the users answer a series of preset general body shape-related descriptive questions (“yes” or “no”), and utilize the mapping relationship from Step (2) to rapidly output accurate human body parameters in line with the actual situation of the users.

In the said Step (1), the accurate data of the human body's different parts are within a certain range; with male neck shape as an example, the general body shape description is set in the converting program: when the neck circumference inputted is not more than 35 cm, the neck shape is “slightly thin”; when falling within 35-40 cm, the neck shape is “normal”; when greater than 40 cm, the neck shape is “slightly thick”. Likewise, with male waist shape as an example, the following general body shape description is presented in the converting program: when the waist-to-hip ratio is not more than 0.8, the waist shape is “sunken”; when greater than 0.8 and not more than 0.87, the waist shape is “straight”; when greater than 0.87 and not more than 0.93, the waist shape is “generally protruding”. In this way, all human body parameters inputted can be converted to get a group of general body shape descriptions about the human body model, namely, a group of answers to the body shape-related descriptive questions.

(1-1) For a certain group of human body measurements, a group of general human body descriptions can be outputted with the help of the converting program, such as “normal” neck shape, chest shape with “severely muscular”, “regular” shoulder shape, “straight” back, “slightly short” arm length, “generally protruding” waist shape, “flat” abdomen shape, “inverted triangular” body shape, “medium-sized” skeleton, and “normal” leg shape.

(1-2) When the model is being used in real life, the user shall answer a group of predefined body shape-related descriptive questions to get general body shape descriptions about the user.

In the said Step (3), every 3D human body model is equipped with a group of human body measurements; to get a 3D human body model in line with the user's real body shape, general body shape descriptions given by the user shall be correlated with human body measurements of corresponding body shapes, which are called mapping relationship.

Above are detailed descriptions about this invention, but the embodiments of this invention are not limited to the above ones, and other alterations, replacements, combinations, and simplifications made under the guidance of the core idea of this invention shall also be included in the protection range of this invention.

What is claimed is:

1. A method of automatically generating human-body parameters using machine learning, comprising the following steps:
(1) initializing a converting program, inputs of which are accurate human-body parameters and outputs of which are general body-shape descriptions; inputting several groups of accurate human-body parameters into the converting program, so as to obtain various combinations of general body-shape descriptions, which are to be used as training sets for subsequent steps; wherein the accurate human-body parameters comprise gender, height, weight, neck circumference, shoulder width, chest circumference, waist circumference, arm length, front waist length, and hip circumference; and the general body shape descriptions comprise descriptions of neck type, shoulder type, back, chest type, arm length, waist type, abdomen type, and body type; wherein
(i) the description of the neck type is divided as follows:
if the neck circumference≤35 cm, it is described as slim;
if 35 cm<neck circumference<40 cm, it is described as normal; and
if the neck circumference≥40 cm, it is described as thick;
(ii) the description of the chest type is divided as follows:
for males:
if chest circumference/height≤0.55, it is described as flat;
if 0.55<chest circumference/height<0.6 and chest circumference−waist circumference>6 cm, it is described as average muscle;
if 0.55<chest circumference/height<0.6 and chest circumference−waist circumference≤6 cm, it is described as average fat;
if chest circumference/height≥0.6 and chest circumference−waist circumference>10 cm, it is described as significant muscle; and
if chest circumference/height≥0.6 and chest circumference−waist circumference≤10 cm, it is described as significant fat.
for females:
if chest circumference/height≤0.52, it is described as A cup;
if 0.52<chest circumference/height≤0.58, it is described as B cup;
if 0.58<chest circumference/height≤0.64, it is described as C cup;
if 0.64<chest circumference/height≤0.7, it is described as D cup; and
if chest circumference/height>0.7, it is described as E cup;
(iii) the description of arm length is divided as follows:
if arm length/front waist length<1.2, it is described as short;
if 1.2≤arm length/anterior waist length<1.26, it is described as normal.
if arm length/front waist length≥1.26, it is described as long;
(iv) the description of waist type is divided as follows:
if waist circumference/hip circumference≤0.8, it is described as concave;
if 0.8<waist circumference/hip circumference≤0.87, it is described as straight;
if 0.87<waist circumference/hip circumference≤0.93, it is described as protruding; and
if waist circumference/hip circumference>0.93, it is described as significantly protruding;
(v) wherein the description of abdomen type is divided as follows:
for males:
if waist circumference/hip circumference≤0.77, it is described as concave.
if 0.77<waist circumference/hip circumference≤0.87, it is described as flat.
if 0.87<waist circumference/hip circumference≤0.95, it is described as protruding.
if waist circumference/hip circumference>0.95, it is described as significantly protruding.
for females:
if waist circumference/hip circumference≤0.76, it is described as concave.
if 0.76<waist circumference/hip circumference≤0.82, it is described as flat.
if 0.82<waist circumference/hip circumference≤0.92, it is described as protruding.
if waist circumference/hip circumference>0.92, it is described as significantly protruding.
(vi) the description of body type is divided as follows:
for males:
if chest circumference−waist circumference<2 cm, it is described as an O shape;
if 2 cm≤chest circumference−waist circumference<5 cm, it is described as an equilateral triangle shape;
if 5 cm≤chest circumference−waist circumference<10 cm, it is described as an H shape;
if chest circumference−waist circumference≥10 cm, it is described as an inverted triangle shape;
for females:
if 0.39<shoulder width/hip circumference≤0.49 and (chest circumference+hip circumference)/(2×waist circumference)≤1.12, it is described as an O shape;
if 0.39<shoulder width/hip circumference≤0.49 and 1.12<(chest circumference+hip circumference)/(2×waist circumference)≤1.2, it is described as an H shape;
if shoulder width/hip circumference>0.49, it is described as an equilateral triangle shape;
if 0.39<shoulder width/hip circumference≤0.49 and 1.2<(chest circumference+hip circumference)/(2×waist circumference), it is described as an X shape;
if shoulder width/hip circumference<0.39, it is described as an inverted triangle shape; and
all human-body parameters inputted are converted to obtain a group of the general body-shape descriptions about the 3D human body model, wherein the group of the general body-shape descriptions are a group of answers to the descriptive questions about body shape;
(2) carrying out training through machine learning by using the training sets obtained from Step (1), to obtain a mapping relationship between the general body-shape descriptions and parameters of a 3D human body model.

2. The method of claim 1, wherein for a certain group of human body measurements, the group of general human body descriptions are outputted with the help of the converting program.

3. The method of claim 2, wherein the user answers a group of predefined body shape-related descriptive questions to obtain general body shape descriptions about the user.

4. The method of claim 3, wherein the 3D human body model further comprises a group of human body measurement data; general body-shape descriptions given by the user are correlated with human body measurement data of a corresponding body shape to obtain a 3D human body model in line with the user's real body shape.

5. The method of claim 1, wherein the specific steps of step (2) are as follows:
(2-1): dividing the several groups of the accurate human-body parameters inputted in step (1) and corresponding general body shape descriptions into training and testing sets;
(2-2) using the general body shape descriptions from the training set as inputs and the accurate human body parameters as outputs, training multiple regression decision tree models with different hyperparameters; wherein these models represent the mapping relationships between the general body shape descriptions and the accurate human body parameters;
(2-3) testing the trained regression decision tree models with the testing set data, and calculating the corresponding linear regression coefficient R2 using the following formula:
$${R}^{2} = 1 - \frac{SSE}{SST} ;$$
$$SSE = {\sum }_{i = 1}^{n} {( {y}_{i} - {f}_{i} )}^{2} ; and$$
$$SST = {\sum }_{i = 1}^{n} {( {y}_{i} - \overline{y} )}^{2}$$
where SSE represents the sum of squared residuals, SST represents the total sum of squares, yi represents the i-th group of true precise human body parameters, fi represents the i-th group of predicted precise human body parameters, and represents the average value of all true precise human body parameters in the testing set;
(2-4) selecting the regression decision tree model with the highest R2 value as the final prediction model, which represents the mapping relationship between the general body shape descriptions and precise human body parameters, wherein the ratio of data quantity between the training set and the testing set in step (2-1) is 7:3, and
recording gender, height, and weight information from a user and the user's responses to a series of preset general descriptive questions about body shape, and using the mapping relationship obtained from Step (2), to output the accurate human-body parameters representing an actual human body of the user.
